(19) United States
(12) Patent Application Publication
(10) Pub. No.: US 2024/0315766 A1
(43) Pub. Date: Sep. 26, 2024

(54) DILATOR WITH BUILT-IN BALLOON FOR USE DURING TRANSSEPTAL PROCEDURE

(71) Applicant: Boston Scientific Scimed, Inc., Maple Grove, MN (US)

(72) Inventor: Spencer FODNESS-BONDHUS, Columbia Heights, MN (US)

(21) Appl. No.: 18/608,757

(22) Filed: Mar. 18, 2024

Related U.S. Application Data
(60) Provisional application No. 63/453,375, filed on Mar. 20, 2023.

(51) Int. Cl. A61B 18/14; A61B 17/00; A61B 17/34; A61B 18/00; A61M 25/10

(52) U.S. Cl. CPC A61B 18/1492; A61B 2017/00247; A61B 2017/3425; A61B 2018/00285; A61B 2018/00601; A61M 25/1018

(57) ABSTRACT

A dilator for facilitating access to a patient's heart and for coupling with a sheath including a sheath hub is disclosed. The dilator includes a dilator body having a proximal end portion and an opposite distal end portion, the distal end portion having a tapered tip portion, the body having a cylindrical wall defining an inner surface and an outer surface, the inner surface defining a dilator lumen extending through an entire length of the body. The dilator further includes an expandable membrane disposed over the body in the distal end portion, the membrane having an inner region in fluid communication with the dilator lumen.

### CROSS REFERENCE TO RELATED APPLICATION

This application claims the benefit of U.S. Provisional Application No. 63/453,375, filed Mar. 20, 2023, the disclosure of which is incorporated herein in its entirety.

### TECHNICAL FIELD

The present invention relates generally to devices and methods for dilation during transseptal procedures. More specifically, the present invention is concerned with a dilator providing additional dilation in facilitating access to a patient's heart.

### BACKGROUND

Devices currently exist for creating a puncture, channel, or perforation within a tissue located in a body of a patient. One such device is the Brockenbrough™ Needle, which is commonly used to puncture the atrial septum of the heart. This device is a stiff elongated needle, which is structured such that it may be introduced into a body of the patient via the femoral vein and directed towards the heart. Occasionally during a transseptal procedure, this device may need to be exchanged for another, larger, transseptal device to supplementally dilate the atrial septum because it may be difficult to get such devices across the septum. The exchange of devices creates longer procedure times, higher risk of complications, among other problems.

Against this background, there exists a continuing need in the industry to provide improved transseptal access system devices and methods. An object of the present invention is therefore to provide such a transseptal access system.

### SUMMARY

In Example 1, a dilator for facilitating access to a patient's heart and for coupling with a sheath including a sheath hub includes a dilator body having a proximal end portion and an opposite distal end portion, the distal end portion having a tapered tip portion, the body having a cylindrical wall defining an inner surface and an outer surface having an outer diameter, the inner surface defining a dilator lumen extending through an entire length of the body. The dilator also includes an expandable membrane disposed over the body in the distal end portion, the membrane having an inner region in fluid communication with the dilator lumen.

Example 2 is the dilator of Example 1 wherein the expandable membrane is configured to transition from a deflated state adapted for delivery through the sheath and an inflated state when positioned distal to the sheath.

Example 3 is the dilator of Example 2 wherein a diameter of the deflated state of the expandable membrane is selected to be slightly smaller than an inner diameter of the sheath through which the dilator is deployed.

Example 4 is the dilator of Example 2 wherein a diameter of the inflated state of the expandable membrane is selected to be larger than an inner diameter of the sheath through which the dilator is deployed.

Example 5 is the dilator of Example 1 wherein the expandable membrane is selected from the group consisting of polyurethane, polyethylene terephthalate (PET), PEBAX, Vestamid, Grilamid, and a combination thereof.

Example 6 is the dilator of Example 1 wherein the dilator lumen is in fluid communication with an external fluid source.

Example 7 is the dilator of Example 6 wherein the external fluid source may be used to inflate and deflate the expandable membrane.

Example 8 is the dilator of any of Examples 1-7 wherein the dilator lumen may deliver the external fluid source to the expandable membrane.

Example 9 is the dilator of Example 1 wherein the dilator further comprises a perforation device disposed within the dilator.

Example 10 is the dilator of Example 9 wherein the perforation device is configured to deliver radiofrequency energy to a tip electrode.

Example 11 is the dilator of any of Examples 1-10 wherein the dilator lumen may house the perforation device in the dilator.

Example 12 is the dilator of Example 1 wherein the expandable membrane is positioned adjacent to and proximal of the beginning of the distal end portion.

Example 13 is the dilator of Example 1 wherein the expandable membrane is positioned on the distal end portion.

Example 14 is the dilator of any of Examples 1-13 wherein the expandable membrane is a balloon or any other expandable mechanism.

Example 15 is the dilator of any of Examples 1-14 wherein the expandable membrane is capable of expanding to an expanded diameter of about double the outer diameter of the dilator.

In Example 16, a dilator for facilitating access to a patient's heart and for coupling with a sheath including a sheath hub includes a dilator body having a proximal end portion and an opposite distal end portion, the distal end portion having a tapered tip portion, the body having a cylindrical wall defining an inner surface and an outer surface having an outer diameter, the inner surface defining a dilator lumen extending through an entire length of the body. The dilator also includes an expandable element disposed about the outer surface, the expandable element is configured to transition from a collapsed state adapted for delivery through the sheath to an expanded state when positioned distal to the sheath.

Example 17 is the dilator of Example 16 wherein the expandable element is an expandable membrane.

Example 18 is the dilator of Example 17 wherein the expandable membrane is selected from the group consisting of polyurethane, polyethylene terephthalate (PET), PEBAX, Vestamid, Grilamid, and a combination thereof.

Example 19 is the dilator of Example 17 wherein the dilator lumen is in fluid communication with an external fluid source.

Example 20 is the dilator of Example 19 wherein the external fluid source may be used to inflate and deflate the expandable membrane.

Example 21 is the dilator of Example 20 wherein the dilator lumen may deliver the external fluid source to the expandable membrane.

Example 22 is the dilator of Example 16 wherein the dilator further comprises a perforation device disposed within the dilator.

Example 23 is the dilator of Example 22 wherein the perforation device is configured to deliver radiofrequency energy to a tip electrode.

Example 24 is the dilator of Example 23 wherein the dilator lumen may house the perforation device in the dilator.

Example 25 is the dilator of Example 17 wherein the expandable membrane is a balloon.

In Example 26, a dilator for facilitating access to a patient's heart and for coupling with a sheath including a sheath hub includes a dilator body defining a dilator lumen adapted to receiving and supporting a puncturing device, the body including a proximal portion for manipulation by a user and a distal portion for placement in or near the heart. The dilator also includes a dilator hub coupled to the proximal portion of the dilator body. The dilator further includes a balloon member attached to the distal portion of the hub and configured to transition from a deflated state adapted for delivery through the sheath and an inflated state when positioned distal to the sheath.

Example 27 is the dilator of Example 26 wherein the balloon member is selected from the group consisting of polyurethane, polyethylene terephthalate (PET), PEBAX, Vestamid, Grilamid, and a combination thereof.

Example 28 is the dilator of Example 26 wherein the dilator lumen is in fluid communication with an external fluid source.

Example 29 is the dilator of Example 28 wherein the external fluid source may be used to inflate and deflate the balloon member.

Example 30 is the dilator of Example 29 wherein the dilator lumen may deliver the external fluid source to the balloon member.

Example 31 is the dilator of Example 26 wherein the dilator further comprises a perforation device disposed within the dilator.

Example 32 is the dilator of Example 31 wherein the perforation device is configured to deliver radiofrequency energy to a tip electrode.

Example 33 is the dilator of Example 32 wherein the dilator lumen may house the perforation device in the dilator.

In Example 34, a method of making a dilator for facilitating access to a patient's heart includes providing a dilator body having a proximal end portion and an opposite distal end portion, the distal end portion having a tapered tip portion, the body having a cylindrical wall defining an inner surface and an outer surface, the inner surface defining a dilator lumen extending through an entire length of the body. The method also includes attaching an expandable membrane disposed over the body in the distal end portion, the membrane having an inner region in fluid communication with the dilator lumen.

Example 35 is the method of Example 34 wherein the expandable membrane is configured to transition from a deflated state adapted for delivery through the sheath and an inflated state when positioned distal to the sheath.

While multiple embodiments are disclosed, still other embodiments of the present invention will become apparent to those skilled in the art from the following detailed description, which shows and describes illustrative embodiments of the invention. Accordingly, the drawings and detailed description are to be regarded as illustrative in nature and not restrictive.

### BRIEF DESCRIPTION OF THE DRAWINGS

FIGS. 1A-1C are schematic illustrations of a medical procedure within a patient's heart utilizing a transseptal access system, according to embodiments of the present disclosure.

FIG. 2 is a schematic illustration of a dilator and radiofrequency perforation device of the transseptal access system illustrated in FIGS. 1A-1C, according to embodiments of the present disclosure.

FIGS. 3A and 3B are schematic illustrations of a distal end portion of a dilator with a built-in expandable membrane for use in the transseptal access system of FIGS. 1A-1C, according to embodiments of the present disclosure.

FIGS. 4A and 4B are schematic illustrations of a distal end portion of a dilator with an expandable element for use in the transseptal access system of FIGS. 1A-1C, according to embodiments of the present disclosure.

While the invention is amenable to various modifications and alternative forms, specific embodiments have been shown by way of example in the drawings and are described in detail below. The intention, however, is not to limit the invention to the particular embodiments described. On the contrary, the invention is intended to cover all modifications, equivalents, and alternatives falling within the scope of the invention as defined by the appended claims.

### DETAILED DESCRIPTION

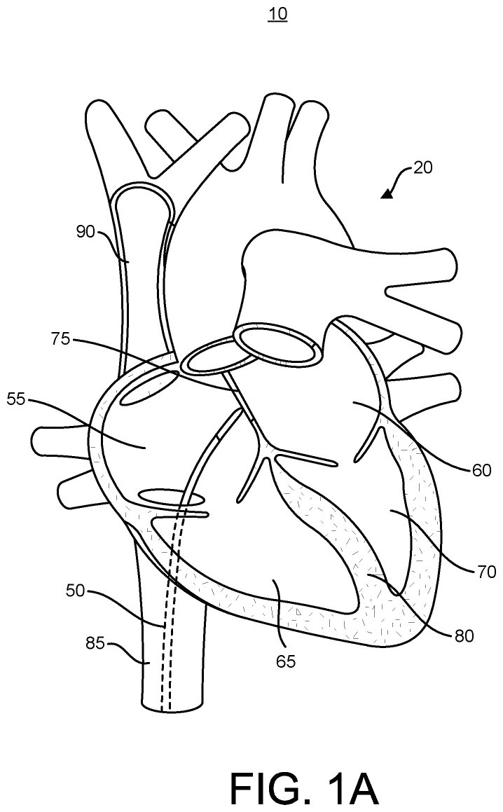

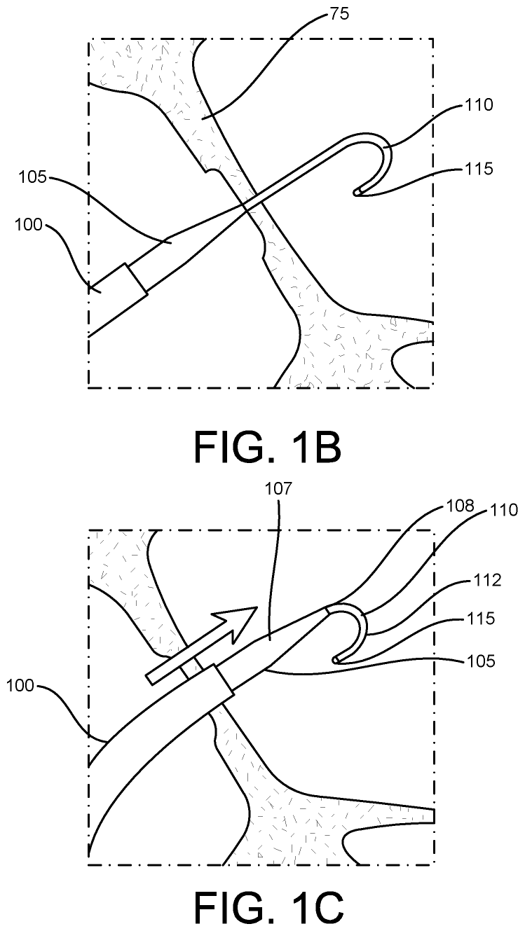

FIGS. 1A-1C are schematic illustrations of a medical procedure 10 within a patient's heart 20 utilizing a transseptal access system 50 according to embodiments of the disclosure. As is known, the human heart 20 has four chambers, a right atrium 55, a left atrium 60, a right ventricle 65 and a left ventricle 70. Separating the right atrium 55 and the left atrium 60 is an atrial septum 75, and separating the right ventricle 65 and the left ventricle 70 is a ventricular septum 80. As is further known, deoxygenated blood from the patient's body is returned to the right atrium 55 via an inferior vena cava (IVC) 85 or a superior vena cava (SVC) 90.

Various medical procedures have been developed for diagnosing or treating physiological ailments originating within the left atrium 60 and associated structures. Exemplary such procedures include, without limitation, deployment of diagnostic or mapping catheters within the left atrium 60 for use in generating electroanatomical maps or diagnostic images thereof. Other exemplary procedures include endocardial catheter-based ablation (e.g., radiofrequency ablation, pulsed field ablation, cryoablation, laser ablation, high frequency ultrasound ablation, and the like) of target sites within the chamber or adjacent vessels (e.g., the pulmonary veins and their ostia) to terminate cardiac arrythmias such as atrial fibrillation and atrial flutter. Still other exemplary procedures may include deployment of left atrial appendage (LAA) closure devices. Of course, the foregoing examples of procedures within the left atrium 60 are merely illustrative and in no way limiting with respect to the present disclosure.

The medical procedure 10 illustrated in FIGS. 1A-1C is an exemplary embodiment for providing access to the left atrium 60 using the transseptal access system 50 for subsequent deployment of the aforementioned diagnostic and/or therapeutic devices within the left atrium 60. As shown in FIGS. 1A-1C, target tissue site can be defined by tissue on the atrial septum 75. In the illustrated embodiment, the target site is accessed via the IVC 85, for example through the femoral vein, according to conventional catheterization techniques. In other embodiments, access to the target site on the atrial septum 75 may be accomplished using a superior approach wherein the transseptal access system 50 is advanced into the right atrium 55 via the SVC 90.

In the illustrated embodiment, the transseptal access system 50 includes an introducer sheath 100, a dilator 105 having a dilator body 107 and a tapered distal tip portion 108, and a radiofrequency (RF) perforation device 110 having distal end portion 112 terminating in a tip electrode 115. As shown, in the assembled use state illustrated in FIGS. 1A-1C, the RF perforation device 110 can be disposed within the dilator 105, which itself can be disposed within the sheath 100. In one embodiment in which the transseptal access system 50 is deployed into the right atrium 55 via the IVC 85, a user introduces a guidewire (not shown) into a femoral vein, typically the right femoral vein, and advances it towards the heart 20. The sheath 100 may then be introduced into the femoral vein over the guidewire, and advanced towards the heart 20. In one embodiment, the distal ends of the guidewire and sheath 100 are then positioned in the SVC 90. These steps may be performed with the aid of an imaging system, e.g., fluoroscopy or ultrasonic imaging. The dilator 105 may then be introduced into the sheath 100 and over the guidewire, and advanced through the sheath 100 into the SVC 90. Alternatively, the dilator 105 may be fully inserted into the sheath 100 prior to entering the body, and both may be advanced simultaneously towards the heart 20. When the guidewire, sheath 100, and dilator 105 have been positioned in the SVC 90, the guidewire is removed from the body, and the sheath 100 and the dilator 105 are retracted so that their distal ends are positioned in the right atrium 55. The RF perforation device 110 described can then be introduced into the dilator 105, and advanced toward the heart 20.

Subsequently, the user may position the distal end of the dilator 105 against the atrial septum 75, which can be done under imaging guidance. The RF perforation device 110 is then positioned such that electrode 115 is aligned with or protruding slightly from the distal end of the dilator 105. The dilator 105 and the RF perforation device 110 may be dragged along the atrial septum 75 and positioned, for example against the fossa ovalis of the atrial septum 75 under imaging guidance. A variety of additional steps may be performed, such as measuring one or more properties of the target site, for example an electrogram or ECG (electrocardiogram) tracing and/or a pressure measurement, or delivering material to the target site, for example delivering a contrast agent. Such steps may facilitate the localization of the tip electrode 115 at the desired target site. In addition, tactile feedback provided by medical RF perforation device 110 is usable to facilitate positioning of the tip electrode 115 at the desired target site.

With the tip electrode 115 and dilator 105 positioned at the target site, energy is delivered from an energy source, e.g., an RF generator, through the RF perforation device 110 to the tip electrode 115 and the target site. In some embodiments, the energy is delivered at a power of at least about 5 W at a voltage of at least about 75 V (peak-to-peak), and functions to vaporize cells in the vicinity of the tip electrode 115, thereby creating a void or perforation through the tissue at the target site. The user then applies force to the RF perforation device 110 so as to advance the tip electrode 115 at least partially through the perforation. In these embodiments, when the tip electrode 115 has passed through the target tissue, that is, when it has reached the left atrium 60, energy delivery is stopped. In some embodiments, the step of delivering energy occurs over a period of between about 1 second and about 5 seconds.

With the tip electrode 115 of the RF perforation device 110 having crossed the atrial septum 75, the dilator 105 can be advanced forward, with the tapered distal tip portion 108 operating to gradually enlarge the perforation to permit advancement of the distal end of the sheath 100 into the left atrium 60.

In some embodiments, the distal end portion 112 of the RF perforation device 110 may be pre-formed to assume an atraumatic shape such as a J-shape (as shown in FIGS. 1B-1C), a pigtail shape or other shape selected to direct the tip electrode 115 away from the endocardial surfaces of the left atrium 60. Examples of such RF perforation devices can be found, for example, in U.S. patent application Ser. Nos. 16/445,790 and 16/346,404 assigned to Baylis Medical Company, Inc. The aforementioned pre-formed shapes can advantageously function to minimize the risk of unintended contact between the tip electrode 115 and tissue within the left atrium 60 and can also operate to anchor the distal end portion 112 within the left atrium 60 during subsequent procedural steps. For example, in embodiments, the RF perforation device 110 can be structurally configured to function as a delivery rail for deployment of a relatively larger bore therapy delivery sheath and associated dilator(s). In such embodiments, the dilator 105 and the sheath 100 are withdrawn following deployment of the distal end portion 112 of the RF perforation device 110 into the left atrium 60. The anchoring function of the pre-formed distal end portion 112 inhibits unintended retraction of the distal end portion 112, and corresponding loss of access to the perforated site on the atrial septum 75, during such withdrawal.

The present disclosure describes novel devices and methods for providing safe transseptal access to the left atrium 60 using radiofrequency energy. As will be explained in greater detail herein, the embodiments of the present disclosure simplify the means of providing electrical connectivity between the radiofrequency puncture device and radiofrequency energy generator, while providing enhanced manipulability by the user.

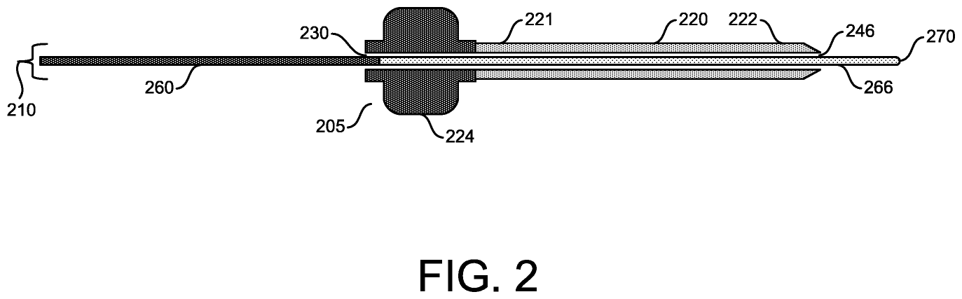

FIG. 2 is an illustration of a dilator 205 and an RF perforation device 210 according to an embodiment of the present disclosure. As shown, the dilator 205 includes a dilator body 220, a dilator hub 224, and a dilator lumen 230 extending longitudinally through the hub 224 and the dilator body 220. Additionally, the dilator body 220 has a proximal end portion 221 and an opposite distal end portion 222 terminating in a distal tip 246. The hub 224 is attached to the proximal end portion 221 of the dilator body 220. While the perforation device in FIG. 2 is described as a radiofrequency perforation device, in embodiments, the perforation device may be a mechanical perforation device.

As can be further seen from FIG. 2, the RF perforation device 210 includes a proximal portion 260 and a distal portion 266 extending from the proximal portion 260 and terminating in a distal functional tip 270 (e.g., a tip electrode such as described above in connection with FIGS. 1A-1C). As will appreciated, the length of the RF perforation device 210 is greater than the length of the dilator 205 so that part of the proximal portion 260 of the RF perforation device 210 extends proximally of the hub 224 when the distal portion 266, particularly the functional tip 270, extends distally of the dilator 205, thus allowing the proximal portion 260 to be manipulated by the user as needed.

In embodiments, when the RF perforation device 210 is in contact with an electrical contact element (not shown) in the hub 224 of the dilator 205, and subsequently to an external RF generator (not shown) capable of generating and delivering radiofrequency energy, the system provides radiofrequency energy to the functional tip 270. In embodiments, the proximal portion 260 of the RF perforation device 210 has an electrically insulated outer surface while the distal portion 266 is electrically conductive and is capable of transferring radiofrequency energy supplied by an external RF generator to the functional tip 270 for subsequent delivery to the target tissue in a transseptal crossing procedure. Additionally, in the illustrated embodiment, the proximal and distal portions 260, 266 are substantially isodiametric, although this is not a strict requirement in all embodiments.

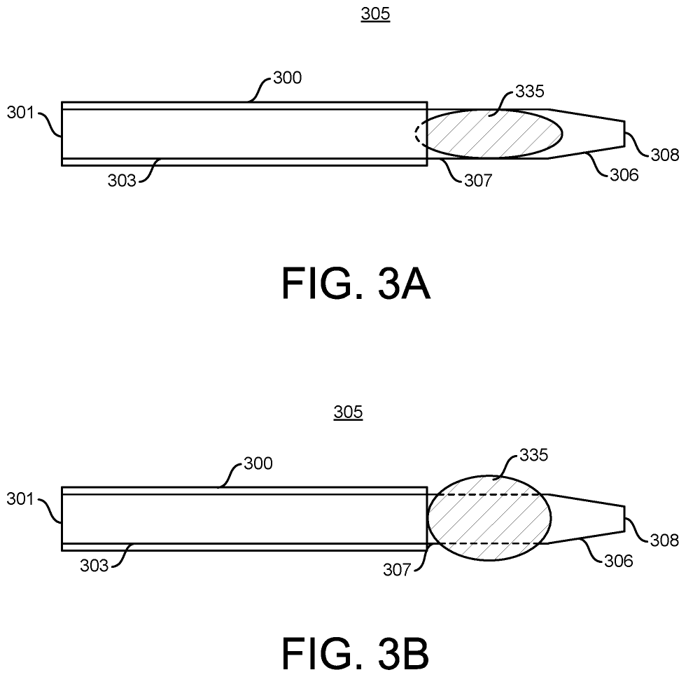

FIGS. 3A and 3B are schematic illustrations of a distal end portion of a dilator 305 with a built-in expandable membrane 335 for use in the transseptal access system 50 of FIGS. 1A-1C, according to embodiments of the present disclosure. As shown, the dilator 305 can be disposed within a sheath 300. The dilator 305 includes a dilator body 307 having a proximal end portion 303 and a tapered distal end portion 306 terminating in a dilator distal tip 308. The dilator body 307 further includes a cylindrical wall defining an inner surface and an outer surface, the inner surface defining a dilator lumen 301 extending through the entire length of the dilator body 307. As further shown, the dilator 305 also includes the expandable membrane 335 disposed over the dilator body 307 proximate the distal end portion 306, the expandable membrane 335 having an inner region in fluid communication with the dilator lumen 301.

In FIGS. 3A and 3B, the expandable membrane 335 is positioned adjacent to and proximal of the beginning of the tapered distal end portion 306, although the specific location of the expandable membrane 335 can be selectively varied to accommodate the particular clinical needs for the dilator 305. In other embodiments, for example, the expandable membrane 335 may be located on the tapered distal end portion 306. In embodiments, the RF perforation device 110 and 210 of FIGS. 1A-1C and FIG. 2 can be disposed within the dilator 305 to provide transseptal access to the left atrium 60 using radiofrequency energy.

The expandable membrane 335 operates as an expandable, FIG. 3B, and collapsible, FIG. 3A, balloon that can be inflated and deflated through the dilator inflation lumen 301 that is in fluid communication with an external fluid source (e.g., a pump or syringe) for inflating and deflating the expandable membrane 335, in a manner known in the art. The expandable membrane 335 can be constructed in any manner and can be made of any materials known in the art relating to balloon catheters such as dilatation balloon catheters, angioplasty catheters, balloon ablation catheters, and the like. Exemplary materials may include polyurethane, polyethylene terephthalate (PET), PEBAX®, Vestamid®, Grilamid®, among others. The particular details of the construction of the expandable membrane 335 are not critical to the embodiments of the disclosure and will be readily ascertained by the skilled artisan from the present disclosure.

In embodiments, the overall diameter of the dilator 305 when the expandable membrane 335 is in a deflated state can be selected to be slightly smaller than the inner diameter of the sheath 300 through which the dilator 305 is deployed, about 2 millimeters (mm) to 10 mm. Furthermore, the expandable membrane 335 can expand to a maximum diameter greater than the sheath 300 diameter when inflated, at least up to 20 mm. In some embodiments, for example, the expandable membrane 335 in the expanded state may be up to double in diameter compared to the collapsible state. In some embodiments, the expandable membrane 335 in the expanded state may be up to triple in diameter compared to the collapsible state. In embodiments, the length of the expandable membrane 335 may be about 5 mm to 20 mm. In some embodiments, the expandable membrane 335 may assume an atraumatic shape such as a balloon shape, as shown in FIGS. 3A and 3B, however, the expandable membrane 335 may take other shapes, such as rounded cones, asymmetric shapes like “acorns” and “turnips,” stepped balloons, expanding baskets, among others.

The expandable membrane 335 in the distal end portion of the dilator 305 can provide a range of functions to the user. In one embodiment, and with reference to FIGS. 1A-1C, the expandable membrane 335 can operate as a stop to delimit distal movement of the dilator 305 into the left atrium 60. In this embodiment, the sheath 300 can be positioned proximally of the expandable membrane 335, which can then be inflated prior to distal advancement of the tapered distal end portion 306 through the atrial septum 75 such that distal advancement of the tapered distal end portion 306 will be impeded once the forward portion of the inflated expandable membrane 335 contacts the wall of the atrial septum 75.

The expandable membrane 335 can also operate to provide supplemental dilation of the perforation through the atrial septum 75 to accommodate deployment of larger diameter therapy sheaths (e.g., sheaths for delivery of ablation catheters, LAA occlusion devices, replacement valves, and the like). For example, in an embodiment, the distal end portion 306 can be advanced through the atrial septum 75 until the expandable membrane 335 is positioned, in a deflated state, within the wall of the septum 75. The expandable membrane 335 can then be controllably inflated to expand the diameter of the perforation to obviate the need to exchange the dilator 305 with a larger diameter dilator as would be necessary to deliver the larger diameter therapy delivery sheath.

In still other embodiments, the inclusion of the expandable membrane 335 may allow the dilator 305 to be used with sheaths 300 of varying diameters, i.e., to effectively vary the maximum diameter of the dilator 305 as needed to accommodate sheaths having a range of inner diameters. In other embodiments, the expandable membrane 335 can assist in the advancement of the sheath 300 across the septum 75.

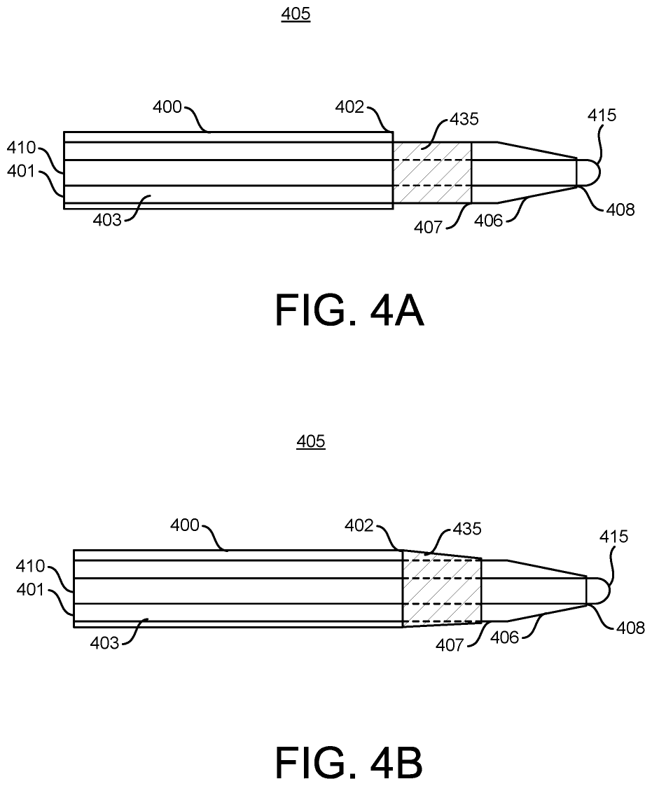

FIGS. 4A and 4B are schematic illustrations of a distal end portion of a dilator 405 with a built-in tapered expandable element 435 for use in the transseptal access system 50 of FIGS. 1A-1C, according to embodiments of the present disclosure. As shown, the dilator 405 can be disposed within a sheath 400 which includes a leading edge 402 at the distal end of the sheath 400. The dilator 405 includes a dilator body 407 having a proximal end portion 403 and a tapered distal end portion 406 terminating in a dilator distal tip 408. The dilator body 407 further includes a cylindrical wall defining an inner surface and an outer surface, the inner surface defining a dilator lumen 401 extending through the entire length of the dilator body 407. As shown, the dilator 405 further includes the expandable element 435 disposed over the dilator body 407 proximate the distal end portion 406, the expandable element 435 having an inner region in fluid communication with the dilator lumen 401. As shown, the expandable element 435 is tapered, which facilitates a smooth transition between the outer surface of the dilator body 407 and the outer surface of the sheath 400.

In FIGS. 4A and 4B, the expandable element 435 is positioned adjacent to and proximal of the beginning of the tapered distal end portion 406, although the specific location of the expandable element 435 can be selectively varied to accommodate the particular clinical needs for the dilator 405. In other embodiments, for example, the expandable element 435 may be located on the tapered distal end portion 406. As shown, a RF perforation device 410 terminating in a tip electrode 415 can be disposed within the dilator 405, which itself can be disposed within the sheath 400.

The expandable element 435 operates as an unexpanded, FIG. 4A, or expanded, FIG. 4B, shoulder that can inflate or deflate in size through the dilator inflation lumen 401 that is in fluid communication with an external fluid source (e.g., a pump or syringe) for inflating and deflating the expandable element 435, in a manner known in the art. In embodiments, the expandable element 435 is an expandable membrane such as, for example, the expandable membrane 335 described above. In embodiments, alternative mechanisms other than the inflatable structure described above may include self-expanding nitinol frames, self-expanding plastic clips, among others.

The expandable element 435 can be constructed in any manner and can be made of any materials known in the art relating to collapsible catheters. In embodiments, the expandable element 435 may be made of polyurethane, polyethylene terephthalate (PET), PEBAX®, Vestamid®, Grilamid®, among others. The particular details of the construction of the expandable element 435 are not critical to the embodiments of the disclosure and will be readily ascertained by the skilled artisan from the present disclosure.

In embodiments, the overall diameter of the dilator 405 when the expandable element 435 is in an unexpanded state can be selected to be slightly smaller than the inner diameter of the sheath 400 through which the dilator 405 is deployed. In embodiments, generally, the diameter of the dilator 405 is in the range of about 2 mm to 10 mm. In further embodiments, the diameter of the expandable element 435 is configured to increase by about 1 mm to 5 mm, when moving from the unexpanded state to the expanded state. As shown, when the expandable element 435 moves from the unexpanded state to the expanded state, the diameter of the expandable element 435 proximal to the leading edge 402 is about equal to the diameter of the leading edge 402, creating a smoother transition for the dilator 405. In embodiments, the length of the expandable element 435 may be about 5 mm to 20 mm. In embodiments, the tapered angle in the expandable element 435 is between 3 to 10 degrees.

The expandable element 435 in the distal end portion of the dilator 405 can provide a range of functions to the user. In one embodiment, the expandable element 435 can operate as a means to make the transition through the atrial septum 75 with the transseptal access system 50 smoother. In the expanded state, FIG. 4B, the diameter at the proximal end of the expandable element 435 equals the diameter of the leading edge 402, creating a flush transition between the sheath 400 and the expandable element 435, which accommodates the advancement of the dilator 405 through the atrial septum 75. In embodiments, this flush transition also accommodates deployment of larger diameter therapy sheaths (e.g., sheaths for delivery of ablation catheters, LAA occlusion devices, replacement valves, and the like). For example, in an embodiment, the distal end portion 406 can be advanced through the atrial septum 75 until the expandable element 435 is positioned, in a deflated state, within the wall of the septum 75. The expandable element 435 can then be controllably inflated to expand the diameter of the perforation to obviate the need to exchange the dilator 405 with a larger diameter dilator as would be necessary to deliver the larger diameter therapy delivery sheath.

In still other embodiments, the inclusion of the expandable element 435 may allow the dilator 405 to be used with sheaths 400 of varying diameters, i.e., to effectively vary the maximum diameter of the dilator 405 as needed to accommodate sheaths having a range of inner diameters.

Various modifications and additions can be made to the exemplary embodiments discussed without departing from the scope of the present invention. For example, while the embodiments described above refer to particular features, the scope of this invention also includes embodiments having different combinations of features and embodiments that do not include all of the described features. Accordingly, the scope of the present invention is intended to embrace all such alternatives, modifications, and variations as fall within the scope of the claims, together with all equivalents thereof.

I claim:

1. A dilator for facilitating access to a patient's heart and for coupling with a sheath including a sheath hub, the dilator comprising:
a dilator body having a proximal end portion and an opposite distal end portion, the distal end portion having a tapered tip portion, the body having a cylindrical wall defining an inner surface and an outer surface having an outer diameter, the inner surface defining a dilator lumen extending through an entire length of the body; and
an expandable element disposed about the outer surface, the expandable element is configured to transition from a collapsed state adapted for delivery through the sheath to an expanded state when positioned distal to the sheath.

2. The dilator of claim 1, wherein the expandable element is an expandable membrane.

3. The dilator of claim 2, wherein the expandable membrane is selected from the group consisting of polyurethane, polyethylene terephthalate (PET), PEBAX, Vestamid, Grilamid, and a combination thereof.

4. The dilator of claim 2, wherein the dilator lumen is in fluid communication with an external fluid source.

5. The dilator of claim 4, wherein the external fluid source may be used to inflate and deflate the expandable membrane.

6. The dilator of claim 5, wherein the dilator lumen may deliver the external fluid source to the expandable membrane.

7. The dilator of claim 1, wherein the dilator further comprises a perforation device disposed within the dilator.

8. The dilator of claim 7, wherein the perforation device is configured to deliver radiofrequency energy to a tip electrode.

9. The dilator of claim 8, wherein the dilator lumen may house the perforation device in the dilator.

10. The dilator of claim 2, wherein the expandable membrane is a balloon.

11. A dilator for facilitating access to a patient's heart and for coupling with a sheath including a sheath hub, the dilator comprising:
a dilator body defining a dilator lumen adapted to receiving and supporting a puncturing device, the body including a proximal portion for manipulation by a user and a distal portion for placement in or near the heart; and
a dilator hub coupled to the proximal portion of the dilator body; and
a balloon member attached to the distal portion of the hub and configured to transition from a deflated state adapted for delivery through the sheath and an inflated state when positioned distal to the sheath.

12. The dilator of claim 11, wherein the balloon member is selected from the group consisting of polyurethane, polyethylene terephthalate (PET), PEBAX, Vestamid, Grilamid, and a combination thereof.

13. The dilator of claim 11, wherein the dilator lumen is in fluid communication with an external fluid source.

14. The dilator of claim 13, wherein the external fluid source may be used to inflate and deflate the balloon member.

15. The dilator of claim 14, wherein the dilator lumen may deliver the external fluid source to the balloon member.

16. The dilator of claim 11, wherein the dilator further comprises a perforation device disposed within the dilator.

17. The dilator of claim 16, wherein the perforation device is configured to deliver radiofrequency energy to a tip electrode.

18. The dilator of claim 17, wherein the dilator lumen may house the perforation device in the dilator.

19. A method of making a dilator for facilitating access to a patient's heart, the method comprising:
providing a dilator body having a proximal end portion and an opposite distal end portion, the distal end portion having a tapered tip portion, the body having a cylindrical wall defining an inner surface and an outer surface, the inner surface defining a dilator lumen extending through an entire length of the body; and
attaching an expandable membrane disposed over the body in the distal end portion, the membrane having an inner region in fluid communication with the dilator lumen.

20. The method of claim 19, wherein the expandable membrane is configured to transition from a deflated state adapted for delivery through the sheath and an inflated state when positioned distal to the sheath.
